(19) United States
(12) Patent Application Publication
(10) Pub. No.: US 2025/0269134 A1
(43) Pub. Date: Aug. 28, 2025

(54) FASTENING A HEATING AND/OR TEMPERATURE MEASURING DEVICE IN AND/OR TO A MEDICAL DEVICE

(71) Applicant: Loewenstein Medical Technology S.A., Luxembourg (LU)

(72) Inventors: Christof GOEBEL, Hamburg (DE); Maike BEER, Hamburg (DE); Henry HAHN, Hamburg (DE); Stefan Hein, Hamburg (DE); Heiko SEPKE, Luetjensee (DE)

(21) Appl. No.: 19/058,188

(22) Filed: Feb. 20, 2025

(30) Foreign Application Priority Data
Feb. 26, 2024 (DE) 102024105354.2

(51) Int. Cl. A61M 16/08; A61M 16/10

(52) U.S. Cl. CPC A61M 16/0816; A61M 16/1075; A61M 2205/3368; A61M 2205/3653

(57) ABSTRACT

A fastening arrangement for a medical device comprises a receiving element which can be fastened to a fastening section of the device; an insertion element which can be inserted into the receiving element and comprises a heating and/or temperature measuring device for heating and/or measuring a temperature of a liquid and/or a gas in a liquid chamber of the device; a connecting element for connecting the insertion element to the receiving element. The connecting element comprises a component that can be moved relative to the receiving element and/or the insertion element between a locking position and an unlocking position, and is configured to lock the insertion element inserted into the receiving element in the locking position, so that removal of the insertion element from the receiving element is not possible.

### CROSS-REFERENCE TO RELATED APPLICATIONS

The present application claims priority under 35 U.S.C. § 119 of German Patent Application No. 102024105354.2, filed Feb. 26, 2024, the entire disclosure of which is expressly incorporated by reference herein.

### BACKGROUND OF THE INVENTION

### 1. Field of the Invention

The present invention relates to a fastening arrangement for fastening a heating and/or temperature measuring device in and/or to a medical device. The invention further relates to a medical device equipped with such a fastening arrangement.

### 2. Discussion of Background Information

A medical device in the form of a respiratory therapy device can be used to treat sleep-related respiratory disorders or other problems with the respiratory pump. A patient is connected to the respiratory therapy device via a suitable patient interface, for example in the form of a face or nose mask, and a patient tube. In order to reduce the risk of pulmonary infections or damage to the lung tissue, it may be necessary to condition the breathing gas supplied to the patient in a suitable way. As a rule, breathing gas conditioning is understood to be the preparation of the breathing gas by heating and/or cleaning the same which, in particular, is important in sensitive patients with damaged lungs. For this purpose, an active breathing gas humidifier—for example a nebulizer, evaporator or bubbler—or a passive breathing gas humidifier, also known as a heat and moisture exchanger or HME for short, can be used.

A breathing gas humidifier can comprise a liquid chamber with heated and/or purified water, over which the breathing gas can be led and thus heated or humidified. The liquid chamber can have an (internal) heating device for heating the water, for example in the form of a heating rod, and/or a temperature measuring device for measuring the temperature of the liquid, for example in the form of a dip tube. External heating devices are also possible.

Such a heating and/or temperature measuring device can be fastened to the liquid chamber, for example via a screw connection.

The breathing gas humidifier should be cleaned regularly since bacteria and fungi can grow in the heated water in the liquid chamber. For regular fundamental cleaning of the liquid chamber and/or of the heating and/or temperature measuring device, it is expedient if the corresponding components can be dismantled by the user, so that the components can be cleaned separately, in particular also including the respective threaded sections.

In order to screw in the heating and/or temperature measuring device so that the resultant screw connection is water-tight or to unscrew it, as a rule both hands are needed. In addition, many users have difficulty estimating the correct torque when screwing together. If the torque is too low, it may occur that water escapes from the liquid chamber because of a leaky screw connection. If the torque is too high, it is possible for damage to the components, in particular plastic parts or the threads, to occur. Many respiratory patients are additionally bodily weakened and/or suffer from additional conditions such as gout, arthritis or rheumatism. Patients with mobility that is restricted in such a way can struggle particularly when assembling and disassembling such a screw-type heating and/or temperature measuring device.

In view of the foregoing, it would be advantageous to have available a fastening arrangement which permits convenient assembly and disassembly of the heating and/or temperature measuring device as compared with screw-type variants.

### SUMMARY OF THE INVENTION

In a first aspect, the invention provides a fastening arrangement for a medical device, in particular a breathing gas humidifier. The fastening arrangement comprises: a receiving element that can be fastened to a fastening section of the medical device; an insertion element that can be inserted into the receiving element and comprises a heating and/or temperature measuring device for heating and/or measuring a temperature of a liquid and/or a gas in a liquid chamber of the medical device; a connecting element for connecting the insertion element to the receiving element, wherein the connecting element comprises a component that can be moved relative to the receiving element and/or to the insertion element between a locking position and an unlocking position. The movable component is designed to lock the insertion element inserted into the receiving element in the locking position, so that removal (or falling out) of the insertion element from the receiving element is not possible, and to release the same in the unlocking position, so that removal is possible.

Such a fastening arrangement also permits patients with restricted mobility simple and trouble-free assembly and disassembly of the heating and/or temperature measuring device, for example for cleaning purposes, without damage or leaks occurring, as is frequently the case in screw-type variants.

The movable component can be designed to produce a form-fitting and/or force-fitting connection between the receiving element and the insertion element inserted into the receiving element when in the locking position, and to release the form-fitting and/or force-fitting connection again in the unlocking position. The form-fitting and/or force-fitting connection can be designed such that manual removal of the insertion element from the receiving element (with a normal expenditure of force) is not possible.

The movable component can be supported on the receiving element and/or on the insertion element and/or on another section of the fastening arrangement and/or the medical device so as to be movable between the locking and unlocking position.

The movable component can be rotatable and/or displaceable relative to the receiving element and/or the insertion element between the locking and the unlocking position and/or follow a straight and/or curved path when moving between the locking and the unlocking position.

For example, the movable component can be permanently connected to the insertion element and, together with the insertion element, can be movable, for example rotatable and/or displaceable, relative to the receiving element between the locking and the unlocking position. Alternatively, the movable component can be permanently connected to the receiving element and, together with the receiving element, can be movable, for example rotatable and/or displaceable, relative to the insertion element between the locking and the unlocking position.

The movable component can be designed to encircle the insertion element inserted into the receiving element, at least in some sections.

The receiving element can, for example, be designed as part of an outer housing of the liquid chamber and/or can be fastened to a section (of an outer housing) of the liquid chamber as the fastening section.

The heating and measurement of the temperature of the gas by means of the heating and/or temperature measuring device may be relevant, for example, when there is a low liquid level in the liquid chamber. It is also conceivable that at least one part of the heating and/or temperature measuring device that is used for the heating or measurement additionally projects into another section of the respective medical device, where this part does not have contact (or at least not completely) with the liquid but with a gas (for example ambient air), so that in this case, additionally or alternatively, this gas is heated or the temperature of this gas is measured.

In a second aspect, the invention provides a medical device, in particular a breathing gas humidifier. The medical device comprises a liquid chamber, a fastening section and a fastening arrangement, as described above and below, the receiving element of which is fastened to the fastening section of the medical device.

The receiving element fastened to the fastening section can be aligned with respect to an opening in a housing of the liquid chamber in such a way that the heating and/or temperature measuring device projects through the opening into the interior of the liquid chamber when the insertion element is inserted completely into the receiving element.

In addition, the medical device can comprise a humidifier, which is designed to humidify breathing gas by using liquid from the liquid chamber and to provide the humidified breathing gas at a connection for a breathing tube.

The liquid chamber can be, for example, a water container and/or have a holding capacity between 100 mL and 1000 mL, between 600 mL and 800 mL or between 300 mL and 400 mL.

The medical device can be, for example, a breathing gas humidifier, a ventilator, in particular in the form of a CPAP and/or high-flow breathing therapy device, or a device for removing secretions.

The medical device can additionally comprise at least one of the following components: a measuring probe for measuring a flow and/or a temperature of a breathing gas; a tube system for conducting a breathing gas to and/or away from a patient; a tube heater for heating one or more tubes of the tube system.

Various embodiments of the invention are described below. These embodiments are not to be understood as restricting the scope of the invention.

According to one embodiment, the movable component can be supported so as to be displaceable between the locking and the unlocking position and/or rotatable through a rotational angle of at most 360°, at most 180°, at most 120° or—preferably—at most 90° about an axis of rotation (for example about a longitudinal axis of the insertion element). In other words, the movement of the movable component between the locking and the unlocking position can be a pure translation, a pure rotation or a combination of a translation and a rotation. The rotation can be limited to a specific maximum rotational angle, for example of at most 360°, at most 180°, at most 120° or—preferably—at most 90°. This can simplify the operation of the movable component, for example in comparison with an embodiment having a screw connection which, as a rule, is tightened by more than one complete revolution.

It is possible that the (for example elongated) insertion element is moved exclusively in a direction parallel to its longitudinal axis during insertion. In other words, the insertion of the insertion element (as opposed to screwing in) can be carried out without changing its angular position or its rotational angle in relation to its longitudinal axis as an axis of rotation.

According to one embodiment, the insertion element can be insertable into the receiving element as far as a sealing position and, in the sealing position, can be connected to the receiving element in a fluid-tight manner, for example in such a way that a gap between the insertion element and the receiving element is sealed off so that no liquid and/or no gas from the liquid chamber can escape to the outside via the gap. In this case, the movable component can be designed to lock the insertion element located in the sealing position in the locking position and/or to unlock the same in the unlocking position. For example, the fastening arrangement can be designed such that in the sealing position the insertion element is forced against the receiving element with a specific force required for the sealing which, for example, can act in the direction of a longitudinal axis of the insertion element.

According to one embodiment, the fastening arrangement can be designed such that the insertion of the insertion element as far as the sealing position is possible without rotating the insertion element and/or the movable component relative to the receiving element by more than 360°, more than 180°, more than 120° or—preferably—more than 90° about an axis of rotation. It is also possible that the insertion of the insertion element as far as the sealing position is carried out by simply pushing it in.

Alternatively, the fastening arrangement can be designed such that, as the insertion element is rotated about the axis of rotation, the receiving element at least partly co-rotates with the insertion element.

As compared with a screw connection, the tightness of which depends significantly on the torque with which the screw connection is tightened, this embodiment has the advantage that neither a relatively high rotational movement nor a noteworthy torque is required to produce the fluid-tight connection. This can highly simplify the assembly and/or make the fastening arrangement more robust against assembly errors.

According to one embodiment, the fastening arrangement can additionally comprise a sealing element and be designed such that as the insertion element is inserted as far as the sealing position, the sealing element is compressed firstly by the insertion element and/or the connecting element and/or the movable component and secondly by the receiving element, in order to connect the insertion element to the receiving element in a fluid-tight manner.

In other words, the sealing element can be designed to close a gap between the insertion element and the receiving element and/or between the connecting element and the receiving element and/or between the movable component and the receiving element in a fluid-tight manner when it is in the compressed state, so that no liquid (or no gas) or no noteworthy quantity of liquid (or of gas) can escape from the fastening arrangement when the insertion element is in the sealing position.

According to one embodiment, the movable component can be designed to apply a force acting in the direction of the sealing position to the insertion element and/or the receiving element when in the locking position. As a result, the sealing action can be improved further. For example, the force can be suitable to compress the sealing element with a defined pressure, so that the fluid-tight connection can be ensured even in the event of relatively large, for example production-induced, assembly-induced or temperature-induced dimensional and/or shape deviations.

According to one embodiment, the insertion element inserted as far as the sealing position can bear on at least one stop. The stop can be designed to prevent further insertion of the insertion element beyond the sealing position. In this way, leaks and/or damage as a result of inserting the insertion element too deeply can be avoided.

According to one embodiment, the movable component can be designed to be springy and/or spring-mounted, for example on the insertion element and/or on the receiving element, in such a way that, in the unlocking position, it has a restoring force acting in the direction of the locking position applied to it. The effect of this is that the movable component moves back into the locking position on its own if it is not otherwise prevented from doing so. This can further simplify the assembly and disassembly. For example, the movable component can be spring-mounted by means of a separate spring element. Additionally or alternatively, the movable component itself can be designed to be elastically deformable in a suitable way and thus permit the sprung mounting.

According to one embodiment, the heating and/or temperature measuring device can comprise at least one of the following constituent parts: a (for example electric) heating element for heating the liquid and/or the gas; a temperature sensor for measuring the temperature of the liquid and/or the gas. Furthermore, the heating and/or temperature measuring device can comprise at least one of the following constituent parts: an electrical contact element for making electrically conductive contact with the (electric) heating element and/or the temperature sensor; a sealing element for sealing off a gap between the receiving element and the insertion element inserted into the receiving element (for example a sealing element as is described above); a blocking element, which is designed to define a maximum insertion depth of the insertion element as it is inserted into the receiving element.

The blocking element, as part of the insertion element, can prevent the user from pushing the insertion element too far into the receiving element. The blocking element can, for example, be designed as an elevated section on the insertion element, which can enclose the insertion element in some sections or completely. As a counterpart to the blocking element, the fastening arrangement in the receiving element can additionally have at least one protrusion for blocking the blocking element as the insertion element is inserted, so that further insertion is no longer possible. The protrusion can be designed, for example, in the form of an elevated ring and/or comprise at least one protuberance. The blocking element can, for example, be the stop described above.

The heating element can be designed to convert electrical current into heat in a controlled manner and to transfer the heat to the liquid and/or the gas in the liquid chamber, for example in order to bring the liquid and/or the gas to a temperature between 20° and 80°. In addition, the heating element can be coupled to a temperature sensor for measuring a current temperature in the liquid chamber and/or be deactivated automatically via a safety circuit when it is detected that the heating element becomes too hot, for example since there is no or too little liquid or no or too little gas in the liquid chamber.

The sealing element can be made, for example, from at least one of the following sealing materials: ethylene propylene diene rubber, chlorobutadiene rubber, natural rubber, ethylene vinyl acetate rubber, styrene butadiene rubber, acrylonitrile butadiene rubber, polyurethane rubber, polytetrafluoroethylene, silicone, silicone foam, an elastomer with a proportion of fiber. The sealing element can be a sealing ring, for example. In this case, the heating and/or temperature measuring device can comprise an annular constriction to receive the sealing ring. The constriction can be formed, for example, as a material cut-out in the insertion element. The sealing element can also be a sealing compound.

At least part of the heating and/or temperature measuring device, for example at least one section of the electrical contact element, can be located outside the liquid chamber when the insertion element is inserted (completely) into the receiving element.

According to one embodiment, at least two of the components or all of the components of the heating and/or temperature measuring device can be arranged one behind another, seen in the direction of the longitudinal axis thereof. The heating and/or temperature measuring device can be elongated, for example rod-like. For example, the sealing element can be arranged between the heating element and the blocking element and/or between the temperature sensor and the blocking element, and/or the blocking element can be arranged between the sealing element and the electrical contact element.

According to one embodiment, the receiving element can have at least one protrusion, which is designed to butt up against the blocking element as the insertion element is inserted into the receiving element, so that further insertion of the insertion element is prevented.

According to one embodiment, the receiving element can be formed as a channel, in particular as a cylindrical channel, having an insertion side, a limiting side, a casing and an inner wall. In this case, the protrusion can be arranged on the limiting side and/or on the inner wall. “Casing” can be understood, for example, as an element which envelops a longitudinal section of the channel in the direction of its circumference, at least in some sections. The casing can also envelop the longitudinal section completely in the direction of its circumference and/or, for example, be designed in the form of a ring, shell and/or collar. “Inner wall” can, for example, be understood as at least one section of an inner lateral surface of the channel. The channel can, for example, have a cross section the same as or similar to the insertion element.

For example, the two cross sections can be shaped so as to be complementary to each other and/or an internal diameter (an inner lateral surface) of the channel can be slightly larger than an outer diameter (an outer lateral surface) of the insertion element, so that the insertion element can be inserted into the receiving element without any play or with defined play. The insertion element can be insertable into the receiving element from the insertion side. The limiting side—for example seen in the direction of a central or longitudinal axis of the channel—can be located opposite the insertion side.

According to one embodiment, the connecting element, in particular the movable component, can comprise at least one latching element—for example in the form of a nose or a hook—for latching the insertion element to the receiving element. The latching element can, for example, be designed to latch the insertion element to the receiving element as the movable component is moved into the locking position and/or to release the latching of the insertion element to the receiving element as the movable component is moved into the unlocking position. The latching element can be arranged between the insertion side and the limiting side and/or between the insertion side and the protrusion. In addition, when the insertion element is not inserted, the latching element can form an outlet starting from the inner wall and projecting into the channel and be designed such that as the insertion element is inserted into the receiving element, it is forced into the casing by the insertion element and/or by the manual actuation of a pressure element (for example with a finger) for exerting pressure on the outlet, so that the outlet no longer projects into the channel. For example, a state in which the latching element is accommodated in the casing can correspond to the unlocking position. By contrast, in the locking position the latching element can engage in the insertion element in a form-fitting manner and/or be hooked with the insertion element.

It is possible that the pressure element, together with the latching element, forms the connecting element, in particular the movable component. For example, the pressure element that is connected to the latching element can be actuated by a user during the insertion of the insertion element.

According to one embodiment, the insertion element can further comprise a holding section for holding the insertion element with a hand and/or the connecting element can, for example, comprise the movable component, also a holding section for holding the connecting element or the movable component with a hand. The holding section can be designed such that it is at least partly located outside the receiving element when the insertion element is inserted into the receiving element—for example as far as the sealing position. For example, the holding section can be arranged along the longitudinal axis of the heating and/or temperature measuring device and/or the insertion element beside the electrical contact element and/or between the electrical contact element and the blocking element. A user should be able to safely grip the insertion element and/or the connecting element (for example the movable component) at this holding section without touching the heating and/or temperature measuring device. In addition, the movable component can at least partly surround the holding section of the insertion element and be designed such that a user can also safely grip the movable component.

According to one embodiment, the holding section can comprise: an annular main body to slide onto the insertion element (for example onto the heating and/or temperature measuring device); a holding arm in the radial direction projecting from the main body for moving (for example rotating and/or displacing) the main body relative to the insertion element (and/or to the receiving element).

The holding arm can have, for example at its free end, a flat grip element for gripping the holding arm with a thumb and index finger. In other words, the holding arm can, for example, be designed in the manner of a flag. It is possible that the connecting element and/or the movable component is/are at least partly formed by the main body. An appropriate warning and/or operating instructions can be attached on and/or to the holding section, preferably on and/or to the holding arm and/or the flat grip element. Such a holding section forms a particularly long lever arm for locking and unlocking the insertion element and/or for pressing on the sealing element. Furthermore, such a holding section can function as a particularly easily readable, unique position indicator for the locking and unlocking position. This can improve the operating convenience further.

In addition, the holding section can be movable relative to the insertion element and/or the receiving element between a blocking position and a release position, and can be designed to block a mechanical coupling of the medical device, for example a ventilator, to a further device, for example a breathing air humidifier, when in the blocking position and to permit the coupling when in the release position. It is possible that the release position corresponds to the locking position and/or the blocking position corresponds to the unlocking position. In other words, the holding section can be designed such that the mechanical coupling can be made only when the insertion element is locked.

According to one embodiment, the fastening arrangement can be designed such that, to connect the insertion element to the receiving element via the connecting element, an insertion movement, a sealing and a closing movement are made. With the insertion movement, the insertion element can be inserted into the receiving element, with the sealing, the insertion element can be connected to the receiving element in a fluid-tight manner and, with the closing movement, the insertion element can be locked in the receiving element. The closing movement can additionally comprise a relative movement of the movable component relative to the insertion element and to the receiving element.

According to one embodiment, the insertion movement can be made at least partly in a direction parallel to a longitudinal axis of the heating and/or temperature measuring device and/or of the insertion element.

According to one embodiment, the sealing can be carried out by the insertion movement.

According to one embodiment, the sealing can be carried out in a manner decoupled from the closing movement.

According to one embodiment, the closing movement can at least partly be made in a direction at an angle to or transverse to a longitudinal axis of the heating and/or temperature measuring device and/or the insertion element.

According to one embodiment, the closing movement can comprise a rotation of the movable component relative to the insertion element and/or the receiving element about a longitudinal axis of the heating and/or temperature measuring device and/or the insertion element by a rotational angle between 30° and 180°, preferably between 45° and 120°, particularly preferably between 80° and 100° or between 60° and 100°. The closing movement can follow a straight and/or curved path.

The sealing can, for example, be carried out solely by the insertion movement along the longitudinal axis. The fluid-tight connection of the insertion element to the receiving element can also be produced by the insertion element being inserted into the receiving element (as far as the stop in an axial end position) and by an axial force applied during the insertion being applied to the sealing element. Thus, no additional (axial) force is required for the sealing. The closing movement can be carried out after the sealing. In particular, a gap between the receiving element and the insertion element located in the axial end position can be sealed off independent of any rotation of the insertion element about its longitudinal axis.

According to one embodiment, the fastening arrangement can further comprise a bayonet fitting for locking the insertion element in the receiving element, in particular in the locking position. The bayonet fitting can comprise (at least) one male element and (at least) one female element complementary to the male element as parts of the bayonet fitting. The connecting element, in particular the movable component, can comprise a first of the parts of the bayonet fitting. Additionally or alternatively, the receiving element can comprise a second of the parts of the bayonet fitting.

To lock the bayonet fitting, the male element can be moved relative to the female element, in which the male element engages, from the unlocking to the locking position, the male element following the course of the female element. As a rule, such a movement requires considerably less expenditure of force than the rotational movement when tightening a screw.

For example, the female element can be formed by at least one groove and/or the male element by at least one knob. “Knob” can generally be understood as an elevation, in particular a round elevation. The groove or the grooves, seen in the longitudinal direction, can extend in the form of a spiral in the direction of the protrusion, for example in a way similar to the furrows of a screw thread with a certain pitch. The groove or the grooves can each enclose an angle of 90° or less, preferably of 45° or less, with a plane through the insertion side. The female element can, for example, be arranged on the insertion side of the channel.

According to one embodiment, the connecting element, in particular the movable component, can be designed as a union element. The union element can have a union sleeve placed on the insertion element and rotatable and/or displaceable relative to the insertion element, which sleeve can form a grip element, and comprise the first part of the bayonet fitting. Thus, an undesired relative movement of the (compressed) sealing element in relation to the heating and/or temperature measuring device can be avoided. Under certain circumstances, such a relative movement can lead to the sealing element slipping or wearing excessively, which can lead to leaks. The union sleeve can in particular be mounted such that it can be rotated and/or displaced between the locking and the unlocking position and/or on the insertion element.

For example, the union sleeve can have two knobs on mutually opposite sides as male elements of the bayonet fitting.

According to one embodiment, the union sleeve can be placed on the holding section, in particular in such a way that it is at least partly located outside the receiving element when the insertion element is (for example completely) inserted into the receiving element. The union sleeve can be mounted on the insertion element such that it can be rotated and/or displaced relative to the holding section.

According to one embodiment, the union element can be mounted such that it can be displaced relative to the holding section along a longitudinal axis of the heating and/or temperature measuring device and/or the insertion element. In this case, at least one holding element can be formed on the side of the union element that faces the holding section, in order to limit a travel of the union element as it is displaced in at least one direction along the longitudinal axis.

### BRIEF DESCRIPTION OF THE DRAWINGS

Embodiments of the invention are described below with reference to the appended drawings. Neither the description nor the drawings are to be understood as restricting the scope of the invention. In the drawings:

FIG. 1 shows a fastening arrangement according to one embodiment of the invention.

FIG. 2 shows a fastening arrangement according to a further embodiment of the invention.

FIG. 3 shows a receiving element of a fastening arrangement according to one embodiment of the invention.

FIG. 4 shows an insertion element of a fastening arrangement according to one embodiment of the invention.

FIG. 5 shows a receiving element of a fastening arrangement according to a further embodiment of the invention.

FIG. 6 shows an insertion element of a fastening arrangement according to a further embodiment of the invention.

FIG. 7 shows a cross-section through the receiving element from FIG. 5.

The figures are purely schematic and not to scale. If the same designations are used in various drawings, then these designations designate the same or equivalent features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

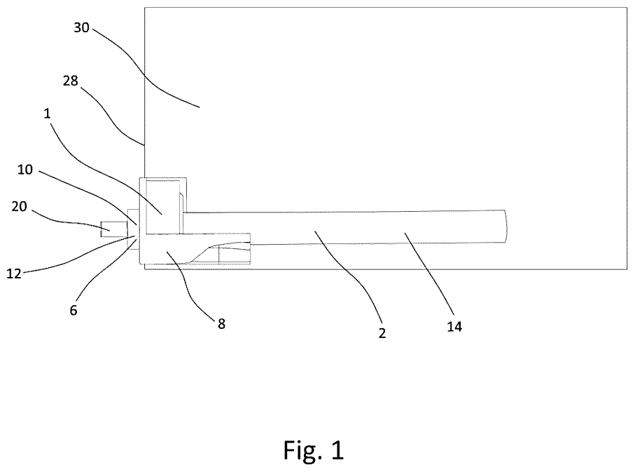

FIG. 1 shows a fastening arrangement 1 for a heating device 2 in a medical device (not shown here). The fastening arrangement 1 comprises an insertion element 6, a receiving element 8 and a connecting element 10 for connecting the insertion element 6 and the receiving element 8. The receiving element 8 is formed as part of an outer housing 28 of a liquid chamber 30, wherein the liquid chamber 30 is designed as a humidifying device for a respiratory therapy device (not shown here). The insertion element 6 in the variant shown comprises a heating device 2, which in turn comprises a heating element 14 and an electrical contact element 20. The receiving element 8 is designed such that it can at least partly accommodate the insertion element 6, wherein, here, the heating device 2 itself and the electrical contact element 20 are located outside the receiving element 8. The connecting element 10 has a component 12 which can move relative to the insertion element 6 and the receiving element 8 and which, when inserted, encircles the insertion element 6, at least in some sections.

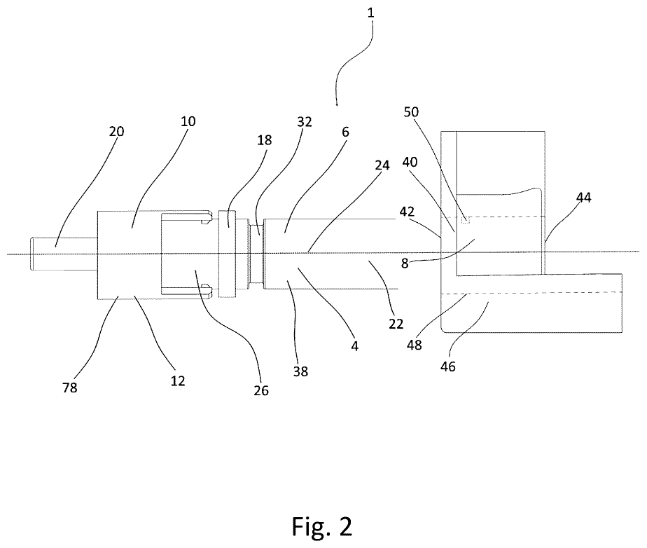

FIG. 2 shows an alternative embodiment of the fastening arrangement 1. To make it easier to see the individual components, the insertion element 6 and the receiving element 8 are shown in a detached, i.e. disconnected, state here. The receiving element 8 has a cylindrical channel 40, in which at least some sections of the insertion element 8 are located following the insertion. In addition, the receiving element 8 has an insertion side 42, which faces the insertion element 6 during insertion, and a limiting side 44, which faces away from the insertion element 6. The channel 40 has an inner wall 48 and a casing 46. Formed on the inner wall 48 in the vicinity of the limiting side 44 is a protrusion 50, which is intended to block the insertion element 6 during insertion, in particular the blocking element 18 of the insertion element 6, so that further insertion along the longitudinal axis 24 is no longer possible. The insertion element 6 comprises a temperature measuring device 4 which, along the longitudinal axis 24, has an electrical contact element 20, a holding section 26, a blocking element 18 and a temperature sensor 22. A constriction 32 between the blocking element 18 and the temperature sensor 22 is designed to accommodate a sealing element 16, in particular a sealing ring 34 (not shown here). Attached to the holding section 26 is the connecting element 10 which, here, is designed as a component 12 that can be moved around the holding section 26 and which encircles the insertion element 6 at least in some sections. The movable component 12 is at the same time a grip element 78, at which a user can safely grip the insertion element 6.

As the insertion element 6 is inserted into the receiving element 8, the connecting element 10 connects the components in such a way that an insertion movement, a sealing and a closing movement are carried out. The closing movement comprises a relative movement of the movable component 12, here a rotational movement. The insertion movement is carried out along the longitudinal axis 24 here. The closing movement is carried out orthogonally to the longitudinal axis 24. The sealing via the sealing element 16 (not shown) is substantially carried out by the insertion movement along the longitudinal axis 24 and is substantially decoupled from the closing movement.

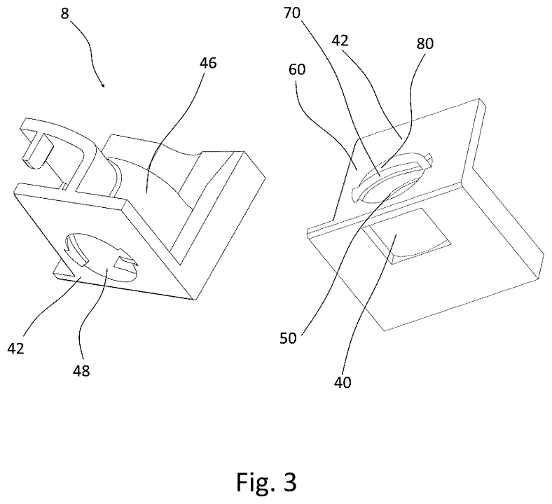

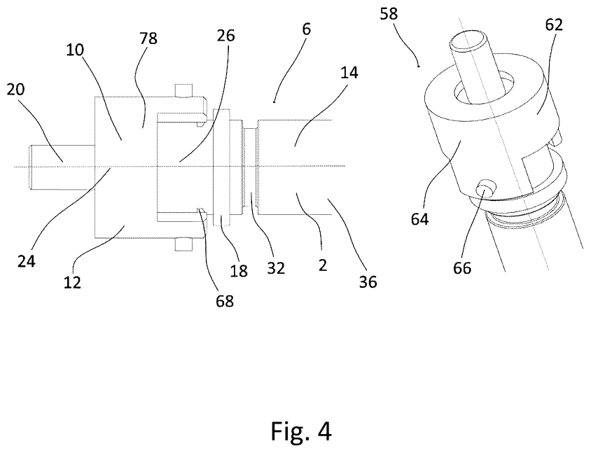

FIG. 3 shows a first embodiment of the receiving element 8. The receiving element 8 in the variant shown here comprises an insertion side 42, which faces the insertion element 6 during the insertion, and a limiting side 44 (not shown here), a channel 40 having an inner wall 48 and a casing 46. Formed on the inner wall 48 is a protrusion 50, which blocks the insertion element 6 during insertion. Part of the receiving element 8 shown forms a female element 60 of a bayonet fitting (counterpart in FIG. 4) with two grooves 70, which extend in the form of a spiral in the direction of the protrusion 50. Each groove 70 encloses an angle 80 which is less than 45° with the insertion side 42.

FIG. 4 shows a first embodiment of the insertion element 6. The movable component 12 forms a male element 58 of the bayonet fitting and corresponds to the receiving element 8 in FIG. 3. The movable component 12 here is formed as a union element 62, which is movably seated on the holding section 26. The union element 62 is formed as an attached, rotatable union sleeve 64 which, at the same time, forms a grip element 78 at which the user can safely grip the insertion element 6. The union sleeve 64 has two knobs 66, which can be accommodated by the grooves 70 in the receiving element 8 (FIG. 3). The union element 62 is mounted such that it can be displaced around the holding section 26 along the longitudinal axis 24. Two holding elements 68 prevent the union element 62 from slipping off the holding section 26 and delimit the displacement along the longitudinal axis 24. The closing movement via the bayonet fitting here comprises a rotational movement about the longitudinal axis 24, the rotation being greater than 90°.

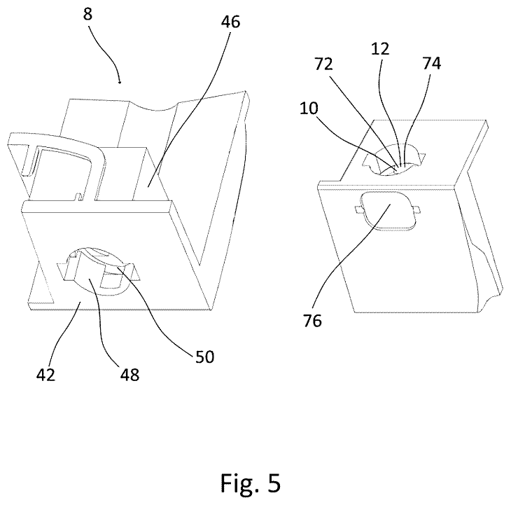

FIG. 5 shows a second embodiment of the receiving element 8. This differs from the variant of the bayonet fitting shown in FIG. 3 and FIG. 4 in that the connecting element 10 in the form of the movable component 12 is part of the receiving element 8. The movable component 12 here is formed as a latching element 72 between the insertion side 42 and the limiting side 44 (not shown here) and, starting from the insertion side 42, is located in front of the protrusion 50. The latching element 72 is designed as an outlet 74 starting from the inner wall 48 and projecting into the channel 40. As the insertion element 6 is inserted into the receiving element 8, the insertion element 6 exerts pressure on the latching element 72, so that the latter is accommodated in the casing 46 and no longer projects into the channel 40. Alternatively or additionally, the pressure on the latching element 72 can be exerted by actuating a pressure element 76, which is connected to the latching element 72 and, together with the latter, forms the connecting element 10, in particular the movable component 12.

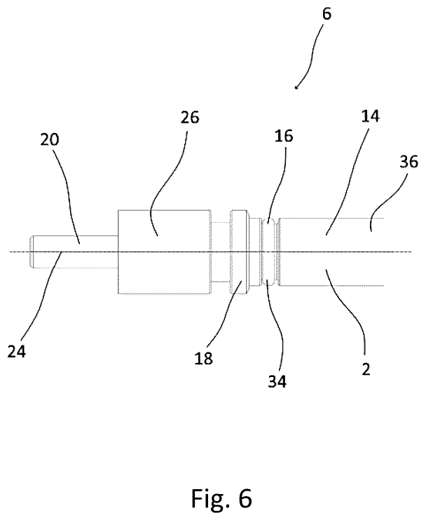

FIG. 6 shows a second embodiment of the insertion element 6. This is compatible with the receiving element 8 shown in FIG. 5. Seen in the direction of the longitudinal axis 24, the insertion element 6 comprises an electrical contact element 20, a holding section 26, a blocking element 18 and a heating element 14, which is formed as a cylindrical heating rod 36 here. Located between the blocking element 18 and the heating element 14 is a sealing element 16, which is formed as a sealing ring 34 here.

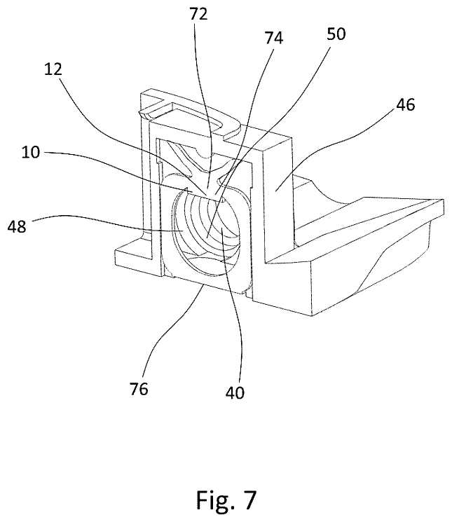

FIG. 7 shows a cross section through the receiving element 8 shown in FIG. 5. Here, the connecting element 10 is formed as a movable component 12. The movable component 12 comprises a latching element 72 in front of the protrusion 50. The latching element 72 is formed as an outlet 74 starting from the inner wall 48 and projecting into the channel 40. In addition, the movable component 12 comprises a pressure element 76, which here is connected to the latching element 72. As a result of pressure on the pressure element 76, the movable component 12 moves into the channel 40 in such a way that the outlet 74 is located flat in the casing 46 and no longer projects into the channel 40.

Finally, it is pointed out that terms such as “have”, “comprise”, “enclose”, “with” etc., do not rule out any other elements or steps, and indeterminate articles such as “a” or “one” do not rule out a multiplicity.

Furthermore, it is pointed out that features or steps which are described with reference to one of the preceding embodiments can also be used in combination with features or steps which are described with reference to other of the preceding embodiments.

Designations in the claims are not to be understood as restricting the scope of the subject matter defined by the claims.

List of reference numerals

| | |
|---|---|
| 1 | Fastening arrangement |
| 2 | Heating device |
| 4 | Temperature measuring device |
| 6 | Insertion element |
| 8 | Receiving element |
| 10 | Connecting element |
| 12 | Movable component |
| 14 | Heating element |
| 16 | Sealing element |
| 18 | Blocking element |
| 20 | Electrical contact element |
| 22 | Temperature sensor |
| 24 | Longitudinal axis |
| 26 | Holding section |
| 28 | Outer housing |
| 30 | Liquid chamber |
| 32 | Constriction |
| 34 | Sealing ring |
| 36 | Heating rod |
| 38 | Dip tube |
| 40 | Channel |
| 42 | Insertion side |
| 44 | Limiting side |
| 46 | Casing |
| 48 | Inner wall |
| 50 | Protrusion |
| 58 | Male element |
| 60 | Female element |
| 62 | Union element |
| 64 | Union sleeve |
| 66 | Knob |
| 68 | Holding element |
| 70 | Groove |
| 72 | Latching element |
| 74 | Outlet |
| 76 | Pressure element |
| 78 | Grip element |
| 80 | Angle of the groove relative to the insertion side |

What is claimed is:

1. A fastening arrangement for a medical device, wherein the fastening arrangement comprises:
a receiving element which can be fastened to a fastening section of the medical device;
an insertion element which can be inserted into the receiving element and which comprises a heating and/or temperature measuring device for heating and/or measuring a temperature of a liquid and/or a gas in a liquid chamber of the medical device;
a connecting element for connecting the insertion element to the receiving element, wherein the connecting element comprises a component that can be moved relative to the receiving element and/or the insertion element between a locking position and an unlocking position, the movable component being configured to lock the insertion element inserted into the receiving element in the locking position, so that removal of the insertion element from the receiving element is not possible, and to release the same in the unlocking position, so that removal is possible.

2. The fastening arrangement of claim 1, wherein the movable component is displaceable between the locking position and the unlocking position and/or is rotatable about an axis of rotation by a rotational angle of at most 360°.

3. The fastening arrangement of claim 1, wherein the movable component is displaceable between the locking position and the unlocking position and/or is rotatable about an axis of rotation by a rotational angle of at most 180°.

4. The fastening arrangement of claim 1, wherein the movable component is displaceable between the locking position and the unlocking position and/or is rotatable about an axis of rotation by a rotational angle of at most 90°.

5. The fastening arrangement of claim 1, wherein the insertion element can be inserted into the receiving element as far as a sealing position and, in the sealing position, is connected to the receiving element in a fluid-tight manner, wherein a gap between the insertion element and the receiving element is sealed such that no liquid and/or no gas can escape to the outside from the liquid chamber via the gap.

6. The fastening arrangement of claim 5, wherein the fastening arrangement is configured such that an insertion of the insertion element as far as the sealing position is possible without rotating the insertion element and/or the movable component relative to the receiving element by more than 360° about an axis of rotation.

7. The fastening arrangement of claim 6, wherein the fastening arrangement further comprises a sealing element for sealing off the gap and is designed such that as the insertion element is inserted as far as the sealing position, the sealing element is compressed firstly by the insertion element and/or the connecting element and/or the movable component and secondly by the receiving element, in order to connect the insertion element to the receiving element in a fluid-tight manner; and/or
the movable component is designed to apply a force acting in a direction of the sealing position to the insertion element and/or the receiving element when in the locking position; and/or
the insertion element inserted as far as the sealing position bears on at least one stop, which is designed to prevent further insertion of the insertion element beyond the sealing position.

8. The fastening arrangement of claim 1, wherein the movable component is designed to be springy and/or is spring-mounted such that, in the unlocking position, it has a restoring force acting in a direction of the locking position applied to it.

9. The fastening arrangement of claim 1, wherein the heating and/or temperature measuring device comprises at least one of the following constituent parts:
a heating element for heating the liquid and/or the gas;
a temperature sensor for measuring the temperature of the liquid and/or the gas;
the heating and/or temperature measuring device further comprising at least one of the following constituent parts:
an electrical contact element for making electrically conductive contact with the heating element and/or the temperature sensor;
a sealing element for sealing off a gap between the receiving element and the insertion element inserted into the receiving element;
a blocking element, which is configured to define a maximum insertion depth of the insertion element as it is inserted into the receiving element.

10. The fastening arrangement of claim 9, wherein the receiving element has at least one protrusion, which is configured to butt up against the blocking element as the insertion element is inserted into the receiving element, so that further insertion of the insertion element is not possible.

11. The fastening arrangement of claim 10, wherein the receiving element is configured as a channel having an insertion side, a limiting side, a casing and an inner wall;
wherein a protrusion is arranged on the limiting side and/or on the inner wall.

12. The fastening arrangement of claim 11, wherein the connecting element comprises at least one latching element for latching the insertion element to the receiving element;
the at least one latching element being arranged between the insertion side and the limiting side and/or between the insertion side and the protrusion;
wherein, when the insertion element is not inserted, the latching element forms an outlet starting from the inner wall and projecting into the channel and is designed such that it is forced, as the insertion element is inserted into the receiving element, into the casing by the insertion element and/or by manual actuation of a pressure element for exerting pressure on the outlet, so that the outlet no longer projects into the channel.

13. The fastening arrangement of claim 1, wherein the insertion element further comprises a holding section for holding the insertion element with a hand, wherein the holding section is configured such that it is at least partly located outside the receiving element when the insertion element is inserted into the receiving element.

14. The fastening arrangement of claim 1, wherein the arrangement further comprises:
a bayonet fitting for locking the insertion element in the receiving element in the locking position, wherein the bayonet fitting comprises a male element and a female element complementary to the male element as parts of the bayonet fitting;
wherein the connecting element comprises a first of the parts of the bayonet fitting;
and/or wherein the receiving element comprises a second of the parts of the bayonet fitting.

15. The fastening arrangement of claim 14, wherein the connecting element is designed as a union element, wherein the union element comprises a union sleeve placed on the insertion element and is rotatable relative to the insertion element, which sleeve forms a grip element, and comprises the first part of the bayonet fitting.

16. The fastening arrangement of claim 15, wherein the union sleeve is placed on the holding section); and or
wherein the union element is mounted such that it can be displaced relative to the holding section along a longitudinal axis of the heating and/or temperature measuring device and/or of the insertion element, wherein at least one holding element is formed on a side of the union element that faces the holding section, in order to limit a travel of the union element as it is displaced in at least one direction along a longitudinal axis.

17. The fastening arrangement of claim 16, wherein the union sleeve is placed on the holding section in such a way that it is at least partly located outside the receiving element when the insertion element is inserted into the receiving element.

18. A medical device, wherein the device comprises:
a liquid chamber;
a fastening section;
the fastening arrangement of claim 1,
the receiving element of the fastening arrangement being fastened to the fastening section.
